(19) United States
(12) Patent Application Publication
(10) Pub. No.: US 2021/0207123 A1
(43) Pub. Date: Jul. 8, 2021

(54) METHOD FOR DETECTING OLIGONUCLEOTIDE CONJUGATES

(71) Applicant: AXOLABS GMBH, Kulmbach (DE)

(72) Inventor: Ingo RÖHL, Memmelsdorf (DE)

(21) Appl. No.: 17/057,914

(22) PCT Filed: May 28, 2019
(86) PCT No.: PCT/EP2019/063806

§ 371 Date: Nov. 23, 2020

(30) Foreign Application Priority Data
May 30, 2018 (EP) 18175099.3

(51) Int. Cl. C12N 15/10; G01N 30/88; B01D 15/32; B01D 15/34; B01D 15/36; G01N 27/447

(52) U.S. Cl. CPC C12N 15/101; G01N 30/88; B01D 15/325; G01N 2030/8827; B01D 15/363; B01D 15/366; G01N 27/44791; B01D 15/34

(57) ABSTRACT

The present invention relates to a method for detecting at least one oligonucleotide conjugate of interest in solution, wherein the oligonucleotide conjugate of interest is composed of a nucleic acid entity and of a nonpolar entity, wherein the nucleic acid entity is chemically linked to the nonpolar entity, and wherein the method comprises the steps of providing a liquid sample comprising the oligonucleotide conjugate of interest; separating the oligonucleotide conjugate of interest from the liquid sample by analytical means under conditions including the presence of at least one cyclodextrine in solution; and detecting the oligonucleotide conjugate of interest by means of qualitative or quantitative analysis.

The high precision analysis of target molecules from biological or liquid samples has developed to be an important tool in various scientific areas including medical or pharmacological diagnostics. Highly sensitive detection systems for the qualitative or quantitative detection and analysis of oligonucleotides are an important tool for state-of-the art analytical laboratories and -analytical applications.

Ion-exchange chromatography in combination with either UV absorbance or fluorescence detection is routinely used in the art for analyzing the degree of purity of synthetic oligonucleotides, or for detecting oligonucleotide modifications. Here, oligonucleotides are separated on a positively charged stationary phase by the number of negative phosphodiester backbone charges which are defined by the length of their backbone. Ion-exchange chromatography coupled with either UV detection or fluorescence readout has further been described in the context of the high resolution analysis of oligonucleotides metabolites (WO 2010/043512 A1).

There is always a need for improved analytical methods in the field of analyzing target molecules such as small molecules, oligonucleotides or oligonucleotide conjugates, in particular in the context of chemical oligonucleotide synthesis quality control.

In the context of the present invention, it has surprisingly been found that the detection, separation and analysis of oligonucleotides can significantly be improved by analytical means in the presence of particular water soluble substances, such as cyclodextrins in solution.

Cyclodextrins are cyclic oligosaccharides consisting of a varying number of alpha-1-4-linked glucose units. These glucose chains create a cone-like cavity into which compounds may enter and form a water-soluble complex, thus altering the physiochemical properties of particular substances such as drugs. 2-hydroxypropyl-beta-cyclodextrin (HP-beta-CD), a hydroxylalkyl derivative of beta-cyclodextrin, has been used as an excipient to improve the solubility of poorly water-soluble drugs (Jiang et al., Journal of Lipid Research, Volume 55 (2014), 1537-1548). Solutions containing cyclodextrins have further been used for the chiral separation of steroid hormone enantiomers in the context of reversed-phase high-performance liquid chromatography (RP-HPLC) (Ye et al., Journal of Chromatography B, 843 (2006) 289-294), or for separating and identifying the four different stereoisomers of methyl jasmonate (Matencio et al., Phytochemical Analysis (2016), wileyonlinelibrary.com). Hence, cyclodextrins have been implicated to improve the purification of small molecules such as stereoisomers.

The analysis and purification of large target molecules such as oligonucleotides, however, significantly differs from the analytics of small molecules and the detection and analysis of oligonucleotides at high resolution is a particular challenge. In the context of the present invention, it has surprisingly been found that the detection and analysis of large target molecules, such as oligonucleotides of a certain length, is significantly improved in the presence of cyclodextrin when used as an additive in solution and when the target molecule is chemically linked to a nonpolar entity, such as a lipophilic or hydrophobic structure which serves as a binding site for cyclodextrin.

In a first aspect, the present invention relates to a method for detecting at least one oligonucleotide conjugate of interest in solution, wherein the oligonucleotide conjugate of interest is composed of a nucleic acid entity and of a nonpolar entity, wherein the nucleic acid entity is chemically linked to the nonpolar entity, and wherein the method comprises the steps of:
a) providing a liquid sample comprising the oligonucleotide conjugate of interest;
b) separating the oligonucleotide conjugate of interest from the liquid sample by analytical means under conditions including the presence of at least one cyclodextrin in solution;
c) detecting the oligonucleotide conjugate of interest by means of qualitative or quantitative analysis.

The term “nucleic acid entity” or “oligonucleotide” as used in the context of the present invention generally refers to any kind of oligomer or polymer composed of either deoxyribonucleotides (DNA) or ribonucleotides (RNA) or both. That is, a nucleic acid entity or an oligonucleotide according to the present invention refers to either a DNA molecule composed of DNA oligonucleotides or to an RNA molecule composed of RNA oligonucleotides or to an oligonucleotide composed of both DNA and RNA nucleotides. The nucleic acid entity or oligonucleotide may be single stranded or in the form of a duplex composed of complementary nucleic acid strands. The nucleic acid entity or the oligonucleotide may also include, but is not limited to, all kind of synthetically designed and/or synthetically manufactured DNA oligonucleotides such as, for example, decoy oligonucleotides. In principle, the nucleic acid entity or the oligonucleotide according to the present invention may include all kind of structures composed of a nucleobase (i.e. a nitrogenous base), a five-carbon sugar which may be either a ribose, a 2′-deoxyribose, or any derivative thereof, and a phosphate group. The nucleobase and the sugar constitute a unit referred to as a nucleoside. The phosphate groups may form bonds with the 2, 3, or the 5 carbon, in particular with the 3 and 5 carbon of the sugar. A ribonucleotide contains a ribose as a sugar moiety, while a deoxyribonucleotide contains a deoxyribose as a sugar moiety. The nucleic acid entity of the invention can contain either a purine or a pyrimidine base or any derivative thereof. The nucleic acid entity or the oligonucleotide according to the present invention, constituted by either ribonucleotides or deoxyribonucleotides or by any combination thereof, may further include one or more modified nucleotide(s). Optionally, the nucleic acid entity or the oligonucleotide may comprise only modified nucleotides. Ribo- and deoxyforms of modified nucleotides may, e.g., include, but are not limited to, 5-propynyl-uridine, 5-propynyl-cytidine, 5-methyl-cytidine, 2-amino-adenosine, 4-thiouridine, 5-iodouridine, N-6-methyl-adenosine, 5-fluorouridine, inosine, 7-propynyl-8-aza-7-deazapurine and 7-halo-8-aza-7-deazapurine nucleosides. The nucleic acid entity or the oligonucleotide as referred to in the context of the present invention may further comprise sugar or ribose modifications such as, e.g., 2′-O-methyl (2′-OMe) RNA or 2′-fluoro (2″-F) RNA. Optionally, the nucleic acid entity or the oligonucleotide of the invention may also or instead comprise one or more modification(s) on the phosphate backbone such as, e.g., phosphorothioates or methyl phosphonates, or any other modification which is known in the art.

The nucleic acid entity can further derive from all kind of natural, non-natural or artificial sources including, but not limited to, viral, bacterial and eukaryotic DNA or RNA. Alternatively, the nucleic acid entity can derive from synthetic sources including the manufacture and/or the chemical synthesis of oligonucleotides for use in research, for use in diagnostics or for use as therapeutic agents. The term “synthesizing” as used herein preferably refers to the manufacture of DNA or RNA oligonucleotides by means of chemical synthesis including, but not limited to, the use of automated DNA and/or RNA synthesizers and/or phosphoramidite chemistry. Automated DNA or RNA synthesizers are routinely used by the person skilled in the art and are commercially available from diverse suppliers such as, e.g., Applied Biosystems (Darmstadt, Germany), Biolytic (Newark, Calif., USA), GE Healthcare or BioAutomation (Plano, Tex., USA).

In a preferred embodiment, the nucleic acid entity of the oligonucleotide conjugate is composed of DNA or RNA nucleotides or any combination thereof. More preferably, the nucleic acid entity is a chemically synthesized oligonucleotide, even more preferably a chemically synthesized oligonucleotide comprising or consisting of modified DNA nucleotides and/or modified RNA nucleotides.

An “oligonucleotide conjugate” according to the present invention refers to a nuclei acid entity or to an oligonucleotide as defined herein, wherein the nucleic acid entity or the oligonucleotide is chemically linked to another substance or to another chemical entity, preferably to a nonpolar entity of any kind. In the context of the present invention, the oligonucleotide conjugate is preferably composed of a nucleic acid entity and of a nonpolar entity, wherein the nucleic acid entity is chemically linked to the nonpolar entity. The chemical linkage of nucleic acid molecules to other chemical entities such as nonpolar entities of any kind is a routine method in the art and well known to the skilled person.

A “nonpolar entity” as referred to herein may be any kind of nonpolar substance, including, but not limited to, any kind of lipophilic, hydrophobic or lipid structure which is suitable for being chemically linked to a nucleic acid entity. That means, in the context of the present invention, the nonpolar entity is selected from the group of those nonpolar substances, nonpolar chemical entities or nonpolar molecules known to the skilled person in the art to be capable of being chemically linked to a nucleic acid entity. The term “nonpolar entity”, therefore, does not extend to those nonpolar molecules or lipid structures which, by their nature or structural features, are not feasible to be used for the purpose of the present invention.

In a preferred embodiment, the nonpolar entity is a lipophilic or a hydrophobic entity. The nonpolar entity is preferably selected from the group consisting of cholesterol, tocopherol and fluoroquinolone. More preferably, the nonpolar entity is cholesterol.

The feature “liquid sample” as used in the context of the present invention refers to all kind of liquid samples containing the target molecule of interest, or, alternatively, a population of target molecules in solution. The liquid sample may be generated by procedures include, but are not limited to, standard biochemical and/or cell biological procedures suitable for the preparation of a cell or tissue extract, wherein the cells and/or tissues may be derived from any kind of organism. A liquid sample according to the present invention may be any kind of buffer, eluent used in the context of analytical means, or, alternatively, a cell extract or a tissue extract derived from a cell or derived from cells grown in cell culture or, alternatively, obtained from an organism by dissection and/or surgery. In particular, a biological sample according to the present invention may be obtained from one or more tissue(s) of one or more patient(s) or from any kind of living human or non-human subject. Preferably, the liquid sample of the present invention is a sample prepared for analytical means and as such no sample directly derived from a living organism, either human or non-human. Provision of a liquid sample from a cell, from a cell extract or, alternatively, from a tissue may include one or more biochemical purification step such as, e.g., centrifugation and/or fractionation, cell lysis by means of mechanical or chemical disruption steps including, for example, multiple freezing and/or thawing cycles, salt treatment(s), phenol-chloroform extraction, sodium dodecyl sulfate (SDS) treatment and proteinase K digestion, or any combination thereof. Equally preferred is that the liquid sample of the invention is provided without any of the herein described precipitation and/or purification steps.

It is to be understood that the term “liquid sample” as used herein generally refers to any kind of aqueous solution, buffer, or liquid solution which allows for the suspension of the target molecules of interest, in particular for the suspension of the oligonucleotide conjugates to be detected.

In a preferred embodiment, the method of the first aspect is characterized in that the analytical means of step b) is selected from the group consisting of anion exchange high performance liquid chromatography (AEX-HPLC), size exclusion liquid chromatography (SEC-LC), reverse phase high performance liquid chromatography (RP-HPLC), ion pairing reversed phase high performance liquid chromatography (IP-RP-HPLC) and capillary gel electrophoresis (CGE).

The analytical means applied to in the context of the methods of the present invention and as set forth above are routine methods commonly used in the art in the field of biochemical analytics and which are well known to the skilled person. The application of diverse analytical means according to the present invention is further exemplified in the example section of this application.

Generally, in the context of the present invention, the oligonucleotide conjugates to be detected, in particular the nucleic acid entity thereof, may have a total length of from 6 to 150 nucleotides, or from 10 to 100 nucleotides, or from 10 to 50 nucleotides, or preferably a length of from 10 to 25 nucleotides. Equally preferred is that the oligonucleotide conjugate of interest has a length in the range of 10 to 80 nucleotides, more preferably in the range of 12 to 50 nucleotides, most preferably in the range of 10 to 40 nucleotides. However, it is evident to the skilled person that the above upper and lower limits may also be combined in order to arrive at different ranges. Moreover, the liquid sample of the invention containing the oligonucleotide conjugate of interest may also contain a population of oligonucleotide molecules with such variable lengths. That is, the sample provided in the context of the present invention may comprise oligonucleotide conjugates of the same length, or may comprise oligonucleotides conjugates of different length, or both. The presence of oligonucleotides or oligonucleotide conjugates of different length, however, does not impair the qualitative or quantitative detection of oligonucleotide conjugates by the methods of the present invention.

In another preferred embodiment, the nucleic acid entity has a length of from 6 to 150 nucleotides, preferably a length of from 10 to 80 nucleotides, more preferably a length of from 12 to 50 nucleotides.

In a preferred embodiment, the method of the first aspect is characterized in that the detecting in step c) includes the detecting of the oligonucleotide conjugate of interest itself as well as the detecting of impurities of the oligonucleotide conjugate. Preferably, these impurities are composed of one or more non-full length nucleic acid entity, preferably in the form of one or more non-full length synthesis product(s) which may be derived from the process of chemical oligonucleotide synthesis. Even more preferred is that the impurities are composed of one or more nucleic acid entities in the form of synthesis product(s) with a length and/or with a structure different to the full-length synthesis product, or any combination thereof.

In the context of the present invention, the terms “detection” or “detecting” generally mean visualizing, analyzing and/or quantifying the target molecule of interest. In particular, the term “detecting” refers to any method known in the art and to the skilled person which is suitable for detecting and analyzing oligonucleotides by means of UV absorption or, alternatively, by means of fluorescence readout. UV absorption is routinely carried out at wavelengths of 254 nm to 260 nm. Methods for the qualitative or quantitative detection of oligonucleotides are well known to the skilled person and have been intensively described in the art. The detecting of oligonucleotide conjugates according to the present invention in the presence and in the absence of at least one cyclodextin is further exemplified by the examples of the present invention.

The term “impurities” as used herein generally means any kind of non-full length oligonucleotide conjugate including any kind of derivative thereof with a nucleic acid entity of either identical, similar, smaller or increased number of nucleotides, resulting in an oligonucleotide conjugate of equal but not necessarily of identical length. The term “equal length”, however, may also include that the oligonucleotide conjugates have an identical length. Equally preferred is that the oligonucleotide conjugates, in particular the nucleic acid entities thereof have a similar length, i.e. a length which slightly differs from the length of the full-length product. An “equal length” according to the present invention, thereby, also includes that the length of the respective nucleic acid entities vary from each other by a couple of nucleotides, preferably by one, two, three, four, five, six, seven, eight, nine, ten or more nucleotides. Alternatively, the oligonucleotide conjugate may also contain or being composed of additives, modifications, or adducts of any kind which may, or may not result, from the process of chemical oligonucleotide synthesis.

Preferably, impurities according to the present invention include, but are not limited to, oligonucleotide conjugates with a nucleic acid entity of similar length which differ in length by only a small number of nucleotides, preferably by a difference of no more than 25 nucleotides in length, more preferably by a difference of no more than 15 nucleotides in length, even more preferred by a difference of no more than 10 or 5 nucleotides in length. The term “impurities” as used herein also includes that the oligonucleotide conjugate(s) of interest and its derivatives may include, comprise or encompass one or more identical or different chemical modification(s). The chemical modifications may be identical or different with respect to both number and/or identity.

In the context of the present invention, is has further been found that carrying out the analytical means of anion exchange high performance liquid chromatography (AEX-HPLC) and alike at particular temperatures results in improved separation profiles. Particular temperature ranges may also be applied to the various other analytical means which have been found suitable for the methods of the present invention. It has further been found that the methods of the present invention, when carried out in the presence of a buffer containing methyl-β-cyclodextrin (MbCD), allow for high resolution results and distinct peaks of cholesterol conjugated oligonucleotides by elution even at ambient temperature.

In a preferred embodiment, the method of the first aspect is characterized in that the analytical means of step b) is selected from the group consisting of anion exchange high performance liquid chromatography (AEX-HPLC), size exclusion liquid chromatography (SEC-LC), reverse phase high performance liquid chromatography (RP-HPLC), ion pairing reversed phase high performance liquid chromatography (IP-RP-HPLC) and capillary gel electrophoresis (CGE), wherein the
i) anion exchange high performance liquid chromatography (AEX-HPLC) is performed at a temperature of from 10° C. to 90° C., preferably at a temperature of from 30° C. to 75° C., more preferably at ambient temperature;
ii) size exclusion high performance liquid chromatography (SEC-HPLC) is performed at a temperature of from 10° C. to 50° C., preferably at a temperature of from 20° C. to 40° C.;
iii) reverse phase high performance liquid chromatography (RP-HPLC) is performed at a temperature of from 10° C. to 100° C., preferably at a temperature of from 40° C. to 70° C.;
iv) ion pairing reverse phase high performance liquid chromatography (IP-RP-HPLC) is performed at a temperature of from 10° C. to 100° C., preferably at a temperature of from 30° C. to 85° C.;
v) capillary gel electrophoresis (CGE) is performed at a temperature of from 10° C. to 60° C., preferably at a temperature of from 30° C. to 50° C.

In another preferred embodiment, the at least one cyclodextrin used in the context of the methods of the present invention is selected from the group consisting of alpha, beta, gamma or delta variants of cyclodextrins. Preferably, the at least one cyclodextrin is selected from the group of beta cyclodextrins. Even more preferred is that at least one cyclodextrin is methyl-beta-cyclodextrin.

In the context of the present invention, it has been found that the presence of at least one cyclodextrin in solution is advantageous for detecting and for analysing an oligonucleotide conjugate of interest in the context of the various analytical means defined herein. The advantageous effects resulting from the presence of at least one cylclodextrin in solution when carrying out the methods for detecting an oligonucleotide conjugate according to the present invention are further exemplified by the examples of the present invention.

In particular, it has been found that a particular final concentration range of cyclodextrin in solution is preferably suitable for obtaining a high peak resolution and, thereby, optimal analytical results.

Preferably, the methods of the present invention are characterized as such that the at least one cyclodextrin in solution is present at a final concentration of from 0.01 mM to 50 mM, preferably at a final concentration of from 0.5 mM to 25 mM, and more preferably at a final concentration of from 1 mM to 15 mM.

Equally preferred is that the at least one cylcodextrin in solution is present at a final concentration of 5 mM, 10 mM or 20 mM.

Preferably, the at least one cyclodextrine is added to the liquid sample before carrying out step b).

Preferably, the detecting in step c) is carried out by means of UV readout, by means of fluorescence readout or by means of mass spectrometry (MS), or any method alike.

In yet another preferred embodiment, the method is used for either analytical or preparative purposes.

In one embodiment, if the method is used for analytical purposes, the quality of the synthesis product is determined in step c), preferably by determining the degree of impurities.

In an equally preferred alternative embodiment, if the method is used for preparative purposes, the yield of the full-length synthesis product is optimized in step c) in that liquid fractions containing the oligonucleotide conjugate of interest are collected. Preferably, the at least one or more liquid fraction(s) which are collected contain a high content of the oligonucleotide conjugate of interest, more preferably characterized in that the oligonucleotide conjugate of interest in the collected fractions comprises a nucleic acid entity of full-size length.

The term “preparative purposes” as used in the context of the present invention generally means any kind of experimental set up in which a high amount of input material is to be purified and/or processed. Generally, a high amount of input material may be any kind of concentration range, preferably any kind of concentration range of between 1 mg and 10 kg of input material. Equally preferred is that the concentration range is even less or more, more preferably up to 20, 30, 50 or 100 kg of input material, given that the experimental setup, in particular the capacity of the purification system is suitable for processing such high amounts of input material.

Input material according to the present invention generally means any kind of synthesis product of interest which is to be detected and/or analysed in the context of the present invention, preferably an oligonucleotide conjugate of interest. The term “high content” or “high amount” as used herein is to be understood as indicating a flexible range of oligonucleotide concentration, preferably a concentration range which reflects a significant amount of the oligonucleotide conjugate input material of interest which is used as a starting material for the analytical means applied in the context of the present invention.

The term “liquid fractions” as used herein generally means any kind of liquid sample which can be derived as an outcome of the analytical means applied in the context of the present invention, preferably in the form of a liquid sample collected from an elution profile, more preferably a liquid sample derived from a chromatographic elution profile. The liquid fraction of the invention can be of any size convenient to the practitioner and/or can be collected by any experimental or practical means available and known to the person skilled in the art.

Preferably, in the context of the present invention, the quality of the synthesis product is defined by the amount and/or by the ratio of the full-length synthesis product versus the amount and/or the ratio of the non full-length synthesis products.

Preferably, the non full-length synthesis products are intermediate and/or irregular synthesis products or a combination of both, more preferably the intermediate synthesis products lack one or more nucleotides at either the 5′- or 3′-end or at both ends. Even more preferred is that the intermediate synthesis products are composed of nucleic acid entities in the form of n-1, n-2, n-3, n-4, n-5, n-6, n-7, n-8, n-9, n-10 with respect to the expected full-length, or alike.

In a further aspect, the present invention pertains to a method for evaluating the quality of a chemical oligonucleotide synthesis product, wherein the method comprises the steps of:
a) providing a liquid sample containing or suspected of containing at least one oligonucleotide conjugate of interest, wherein the at least one oligonucleotide conjugate of interest is composed of a nucleic acid entity and of a nonpolar entity, wherein the nucleic acid entity is chemically linked to the nonpolar entity, and wherein the nucleic acid entity is a chemical oligonucleotide synthesis product;
b) separating the at least one oligonucleotide conjugate of interest from the liquid sample by analytical means under conditions including the presence of at least one cyclodextrine in solution;
c) detecting the at least one oligonucleotide conjugate of interest by means of qualitative or quantitative analysis;
d) collecting liquid fractions;
e) analysing the collected fractions containing or suspected of containing the oligonucleotide conjugate of interest by an analytical means, characterized in that the nucleic acid entity of the oligonucleotide conjugate of interest is composed of the at least one full-length synthesis product.

Evaluating the quality of a chemical oligonucleotide synthesis product according to the present invention generally includes, but is not limited to, the analysis of the degree of purity of the synthesis product, wherein the degree of purity may be determined, but is not limited to, by the analytical means described herein. Evaluating the quality of a chemical oligonucleotide synthesis product also means determining the degree and/or the amount of impurities in the liquid sample, such as, for example, any kind of non-full length synthesis products and/or other synthesis products, such as, for example, any kind of product additives or artifacts. Generally, the degree of purity is the higher, the little impurities are detected. Preferably, the purity of the synthesis product is best, if the at least one or more collected fraction(s) contain at least 75%, more preferably at least 85%, and even more preferred at least 90% of the full-length synthesis product. Optimally, the at least one or more collected fraction(s) contain at least 95% or even 100% of the full-length synthesis product.

Advantages of the methods of the present invention are that the peak width is significantly reduced, that the decrease in peak width results in a significant increase in the resolution of peaks eluting just before and after the main peak, and that the peaks are symmetric in the presence of the at least one cyclodextrin, while they are not in the absence of cyclodextrin as an additive in solution. The improved technical effects of the methods employed in the context of the present invention are further exemplified in the Examples and Figures of the present application.

The method of the second aspect of the present invention is preferably characterized by any one of the embodiments as defined herein, and preferably by any one of the embodiments as defined in the context of the first aspect of the present invention. The embodiments of the methods are further outlined by the examples and figures of the present application.

The term “quantitative readout” generally means all kind of imaging methods known in the art that are suitable to visualize, detect, analyze and/or quantify the oligonucleotides of interest from a sample

Equally preferred is that the detection of the oligonucleotide conjugate is carried out by means of qualitative analysis. Qualitative analysis according to the invention is, for example, exemplified by the examples of the present invention.

Furthermore, detection of the oligonucleotide conjugate may be carried out by quantitative readout. Quantitative readout according to the present invention involves the use of either internal or external standards. Quantitative readout by the use of internal standards has been described in the context of the present invention. Alternatively, and equally preferred is that the quantitative readout involves the use of external standards in form of a comparison to external calibration curves.

Preferably, the external calibration curve is derived from a dilution series of target molecules of known concentration(s) or of know molar weight(s) which are treated under identical conditions as the samples of interest

The following Figures and Examples are intended to illustrate various embodiments of the present invention. As such, the specific modifications discussed therein are not to be understood as limitations of the scope of the invention. It will be apparent to the person skilled in the art that various equivalents, changes, and modifications may be made without departing from the scope of the invention, and it is thus to be understood that such equivalent embodiments are to be included herein.

### FIGURE LEGENDS

FIG. 1: SEC-HPLC Column: GE Healthcare Superdex 75 Increase 10/300 GL. Temperature: Room Temperature 25° C. (non-denaturing: Duplex stays intact during chromatography). Eluent: 1×PBS in 15% ACN with 1 mM Methyl-β-Cyclodextrin or 1×PBS in 15% ACN w/o Methyl-β-Cyclodextrin. Flow rate: 0.9 mL/min. Black trace: Duplex analyzed in presence of Methyl-β-Cyclodextrin. Blue trace: Duplex analyzed in absence of Methyl-β-Cyclodextrin (duplex peak does not elute from column, no peak).

FIG. 2: Single strand analysis of X32755K1 by AEX-HPLC: Column: ThermoFisher Scientific DNA Pac PA200; 4×250 mm Temperature: 85° C. Eluent A: 25 mM TRIS; 1 mM EDTA in 25% Acetonitrile at pH=8; Eluent B 500 mM sodium perchlorate in Eluent A; The eluents are prepared with or without presence of 5 mM Methyl-β-Cyclodextrin. Flow rate: 1.0 mL/min. Compounds are eluted by gradient of eluent B from 24.5% after one minute increased to 37% at 33 minutes. Black trace: X32755K1 analyzed in presence of 5 mM Methyl-β-Cyclodextrin in eluent A and eluent B. Blue trace: X32755K1 analyzed in absence of Methyl-β-Cyclodextrin in eluent A and eluent B.

In the presence of Methyl-β-Cyclodextrin, the main peak is more symmetric, has much smaller peak width at baseline (0.92 vs. 0.62 min) resulting in a greater peak height. Greater peak height corresponds to higher sensitivity for detection of the main peak. Resolution of the impurity peaks from main peak is improved, e.g. the resolution according to the USP (US Pharmacopeia) of later eluting impurity peak to the main peak is increased from 1.48 in absence of Methyl-β-Cyclodextrin to 3.63 in presence of 5 mM Methyl-β-Cyclodextrin in the eluents.

FIG. 3: Single strand analysis of X32755K1 by CGE: Capillary—eCAP DNA Capillary (65 cm total length; 100 μm I.D.), Beckman Coulter, No.: 477477; Temperature: 35° C. Run Buffer: 1×TRIS Borate Buffer with 10 mM Methyl-β-Cyclodextrin or 1×TRIS Borate Buffer w/o 10 mM Methyl-β-Cyclodextrin. Separation Voltage: 30 kV. Blue trace: X32755K1 analyzed in presence of 10 mM Methyl-β-Cyclodextrin. Black trace: X32755K1 analyzed in absence of Methyl-β-Cyclodextrin (single strand peak does not elute from capillary, no peak).

FIG. 4: Single strand analysis of X32755K1 by CGE: Capillary—eCAP DNA Capillary (65 cm total length; 100 μm I.D.), Beckman Coulter, No.: 477477; Temperature: 35° C. Run Buffer: 1×TRIS Borate Buffer with 10 mM Methyl-6-Cyclodextrin or 1×TRIS Borate Buffer with 20 mM Methyl-β-Cyclodextrin or 1×TRIS Borate Buffer w/o 10 mM Methyl-β-Cyclodextrin. Separation Voltage: 30 kV. Pink trace: X32755K1 analyzed in presence of 10 mM methyl-β-cyclodextrin; Blue trace: X32755K1 analyzed in presence of 10 mM Methyl-β-Cyclodextrin. Black trace: X32755K1 analyzed in the absence of methyl-β-cyclodextrin (single strand peak does not elute from capillary, no peak).

FIG. 5: Structure of immobilized cholesterol.

FIG. 6: About 1 mg of crude material was purified via HPLC using the Source 15Q resin at ambient temperature. Buffer contained 30% acetonitrile (ACN).

FIG. 7: About 1 mg of crude material was purified via HPLC using the Source 15Q resin at ambient temperature. Buffer contained 25% ACN and 20 mM Methyl-β-cyclodextrin (MbCD).

FIG. 8: About 100 μg of crude material was purified via HPLC using the Source 15Q resin at 60° C. Buffer contained 30% ACN only, no MbCD was added.

FIG. 9: About 8 mg of crude material was HPLC purified using the TSK Gel resin at ambient temperature. NaBr gradient, 20 mM Na-phosphate, pH 7.8 in 15% ACN containing 20 mM MbCD. Flow rate was 1 mL/min and the gradient was programmed to start from 0% buffer B to reach 40% buffer B in 60 minutes.

FIG. 10: About 8 mg of crude material was HPLC purified using the Source 15Q resin at ambient temperature. NaBr gradient, 20 mM Na-phosphate, pH 7.8 in 15% ACN containing 20 mM MbCD. Flow rate was 1 mL/min and the gradient was programmed to start from 0% buffer B to reach 40% buffer B in 60 minutes.

FIG. 11: About 8 mg of crude material was HPLC purified using the TSK Gel resin at 60° C. Material was eluted using a NaBr gradient, 20 mM Na-phosphate, pH 7.8 in 15% ACN containing 20 mM MbCD. Flow rate was 1 mL/min and the gradient was programmed to start from 0% buffer B to reach 10% buffer B in 5 minutes and subsequently the slope of the gradient was changed to reach 40% B in 60 minutes.

FIG. 12: About 8 mg of crude material was HPLC purified using the Source 15Q resin at 60° C. NaBr gradient, 20 mM Na-phosphate, pH 7.8 in 15% ACN containing 20 mM MbCD. Flow rate was 1 mL/min and the gradient was programmed to start from 0% buffer B to reach 10% buffer B in 5 minutes and subsequently the slope of the gradient was changed to reach 40% B in 60 minutes.

FIG. 13: Analytical results are shown. Pooled fractions correspond to the area of the FLP peak marked with the two-headed arrow (↔) in FIGS. 9 to 12, respectively.

### EXAMPLES

### Example 1: Methyl-β-Cyclodextrin as Additive in SEC-LC

Goal: Development of a SEC-LC method for the analysis of a cholesterol-conjugated oligonucleotide duplex.

Background: Usually, cholesterol-modified oligonucleotides do not elute from an SEC-column. Addition of methyl-β-cyclodextrin to the SEC Buffer masks the cholesterol of the oligonucleotide and thus allows for the compound eluting as a peak from the SEC column.

Test Sample: siRNA-duplex CD-10452K1:

Duplex XD-10452K1

| Abbreviation | Axo ID | Sequence |
|---|---|---|
| FLPs | X32755K1 | 5′-(Chol4)GGAUGAAGUGGAGAUUAGUdTdT-3′ |
| FLPas | X02812K3 | 5′-ACUAAUCUCCACUUCAUCCdTdT3′ |

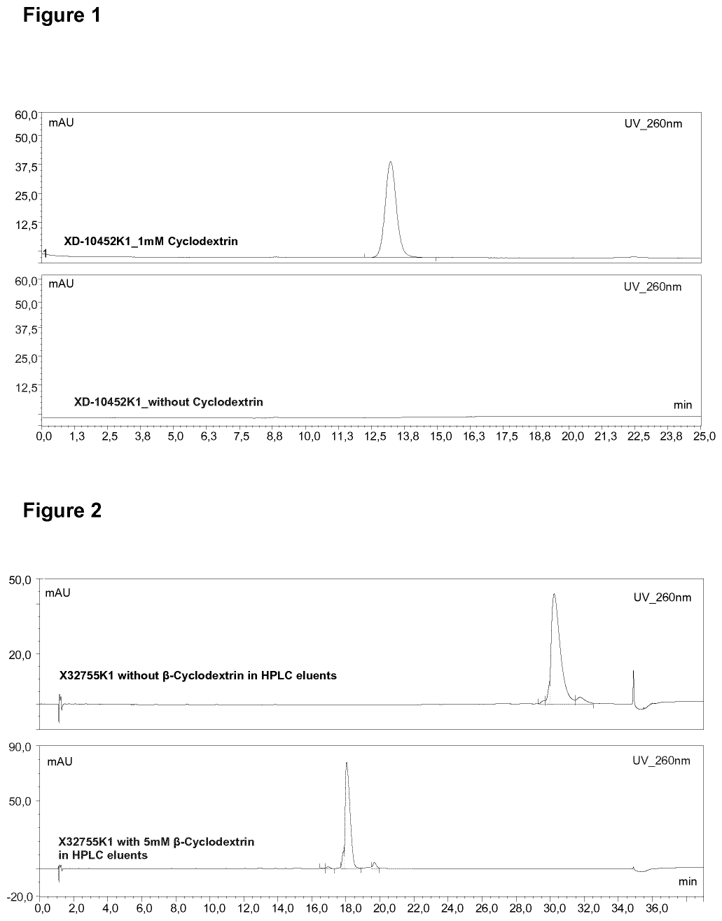

SEC-HPLC Column: GE Healthcare Superdex 75 Increase 10/300 GL. The SEC-LC was performed at room temperature to achieve non-denaturing conditions, so that the siRNA-duplex stays intact during chromatography. The eluents were composed of 1×PBS in 15% ACN with 1 mM methyl-β-cyclodextrin or 1×PBS in 15% ACN without methyl-β-cyclodextrin and a flow rate: of 0.9 mL/min was applied. The result in FIG. 1 shows, that the duplex peak can only be observed in presence of 1 mM methyl-β-cyclodextrin (Black trace), but not in absence (blue trace), as then no peak elutes and the material is strongly bound to the SEC column surface.

### Example 2: Methyl-β-Cyclodextrine as Additive in AEX-HPLC

Goal: Development of AEX-HPLC method for the analysis of cholesterol-conjugated oligonucleotides.

Background: Add Methyl-β-cyclodextrine to the different HPLC Buffers to mask the cholesterol of the oligonucleotide and thus, changing the properties of interaction with the column material.

Test Sample: X32755K1 single stranded oligonucleotide:

| Abbreviation | Axo ID | Sequence |
|---|---|---|
| FLPs | X32755K1 | 5′-(Chol4)GGAUGAAGUGGAGAUUAGUdTdT-3′ |

| | Thermo Fisher Scientific DNAPac PA200, 4 × 250 mm |
|---|---|
| Column | (Thermo; Art. No. 063000) |
| Buffer without beta- | Eluent A: 25%ACN, 1 mM EDTA, 25 mM Tris pH8 |
| cyclodextrin | Eluent B: A with 500 mM NaC104 |
| Buffer with beta- | Eluent A: 25% ACN, 1 mM EDTA, 25 mM Tris pH8 and 5 mM |
| cyclodextrin | cyclodextrine |
| | Eluent B: A with 500 mM NaC104 |
| Column Temp. | 85° C. |
| Flow: | 1.00 ml/min |

TABLE 1
Gradient
Gradient Table

| Time | Flow [ml/min] | % A | % B |
|---|---|---|---|
| −0.5 | 1.0 | 75.5 | 24.5 |
| 0.0 | 1.0 | 75.5 | 24.5 |
| 1.0 | 1.0 | 75.5 | 24.5 |
| 33.0 | 1.0 | 63.0 | 37.0 |
| 33.2 | 1.0 | 0 | 100.0 |
| 33.7 | 1.0 | 0 | 100.0 |
| 34.0 | 1.0 | 75.5 | 24.5 |
| 39.0 | 1.0 | 75.5 | 24.5 |

TABLE 2
Results for AEX-HPLC Analysis of Y32755K1

| | | | |
|---|---|---|---|
| | Peak Width | Relative | Resolution |
| | at baseline | Retention Time | to Main peak |
| Description | [min] | to Main peak | (according to USP) |

AEX-HPLC of X32755K1 with 5 mM cyclodextrine

| | | | |
|---|---|---|---|
| Peak1 | 0.62 | 0.921 | 2.48 |
| Peak2 | 0.36 | 0.940 | 2.46 |
| Peak3 | 1.88 | 0.990 | 0.15 |
| Main Peak | 0.52 | 1.000 | / |
| Peak5 | 0.38 | 1.091 | 3.63 |

AEX-HPLC of X32755K1 without 5 mM cyclodextrine

| | | | |
|---|---|---|---|
| Peak1 | n.a. | 0.983 | n.a. |
| | | (only Peak-Shoulder) | |
| Peak2 | n.a. | 0.991 | n.a. |
| | | (only Peak-Shoulder) | |
| Peak 3 | n.a. | Not detected, | n.a. |
| | | Co-elution | |
| | | with main peak | |
| Main Peak | 0.92 | 1.000 | n.a. |
| Peak4 | 1.11 | 1.050 | 1.48 |

With 5 mM beta-cyclodextrine in AEX-HPLC Buffers the following was observed (FIG. 2). The peak width at baseline is significantly reduced form 0.92 min to 0.52 min. The decrease in peak width results in a significant increase in the resolution of peaks eluting just before and after the main peak. The peaks are symmetric in presence of 5 mM beta-Cylcodextrine and not in the absence of this additive. The results of FIG. 2 show the following:

A) Peak No. 3 is only detected when analyzing in presence of 5 mM beta-cylcodextrine and co-elutes with the main peak in absence of beta-cylcodextrine.
B) Peak No. 2 is resolved with resolution of 2.46 by USP compared to no resolution, as peak only results in a small shoulder on the main peak, but no separation
C) Peak No. 5 is separated with a resolution of 3.68 in presence of 5 mM beta-cylcodextrine and only 1.48 in absence of beta-cylcodextrin.

### Example 3: Methyl-β-Cyclodextrin as Additive in CGE

Goal: Development of a Capillary Gel Electrophoresis Method (CGE) for the Analysis of Cholesterol-conjugated oligonucleotides. All work was conducted on a PA800plus CE instrument from Beckman Coulter. Background: CGE does not work for cholesterol-modified oligonucleotides as compounds are strongly retained by CGE gel and no peaks eluted from the capillary. Addition of 10 mM or more Methyl-β-cyclodextrin to the separation gel and to the separation buffers of the CGE system mobilizes the cholesterol modified strand and sharp peaks can be observed.

Test Sample: single strand X32755K1 (sense strand in AHA1-Duplex XD-10452K1):

| Abbreviation | Axo ID | Sequence |
|---|---|---|
| FLPs | X32755K1 | 5′-(Chol4)GGAUGAAGUGGAGAUUAGUdTdT-3′ |

TABLE 3
Conditions of Capillary Gel Electrophoresis (CGE)

| Capillary | eCAP DNA Capillary (65 cm total length; | |
|---|---|---|
| | 100 pm I.D.), Beckman Coulter, No.: 477477 | |
| Buffer without | 1x TRIS-Borate Buffer | |
| beta-cyclodextrine | | |
| Buffer with | 1x TRIS-Borate Buffer with 10 mM | |
| beta-cyclodextrine | beta-cyclodextrin | |
| Capillary Temp. | 35° | C. |
| Separation Voltage | 30 | kV |

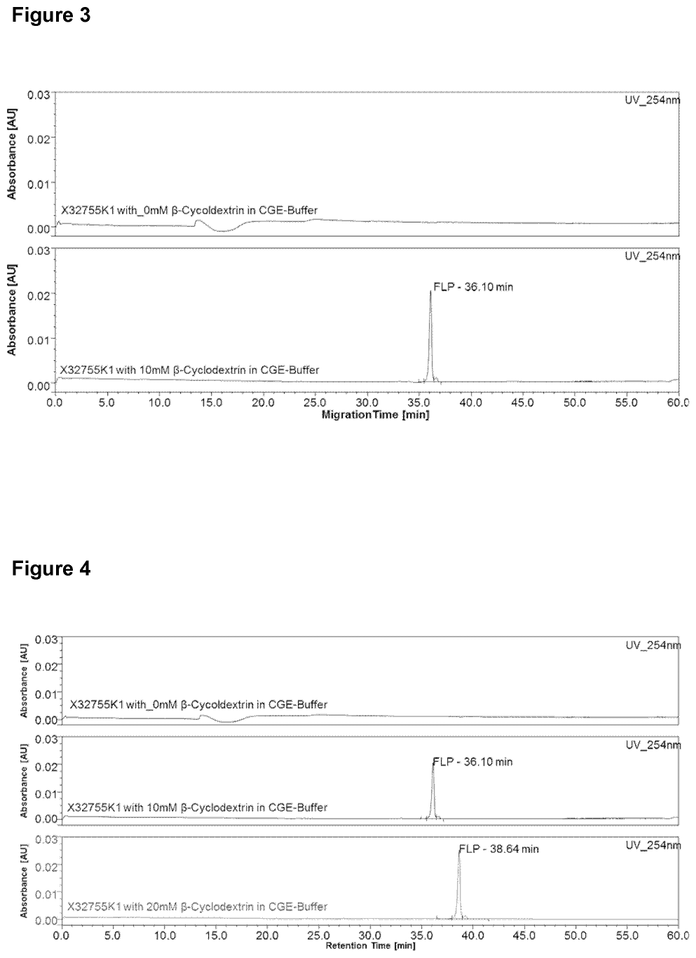

FIGS. 3 and 4 show that X32755K1 can only be analysed in presence of 10 or 20 mM methyl-β-cyclodextrin (blue trace in FIG. 3 and blue and pink trace in FIG. 4), whereas in the absence of methyl-β-cyclodextrin, no peak can be detected.

### Example 4: Methyl-β-Cyclodextrin for IEX HPCL Purifications

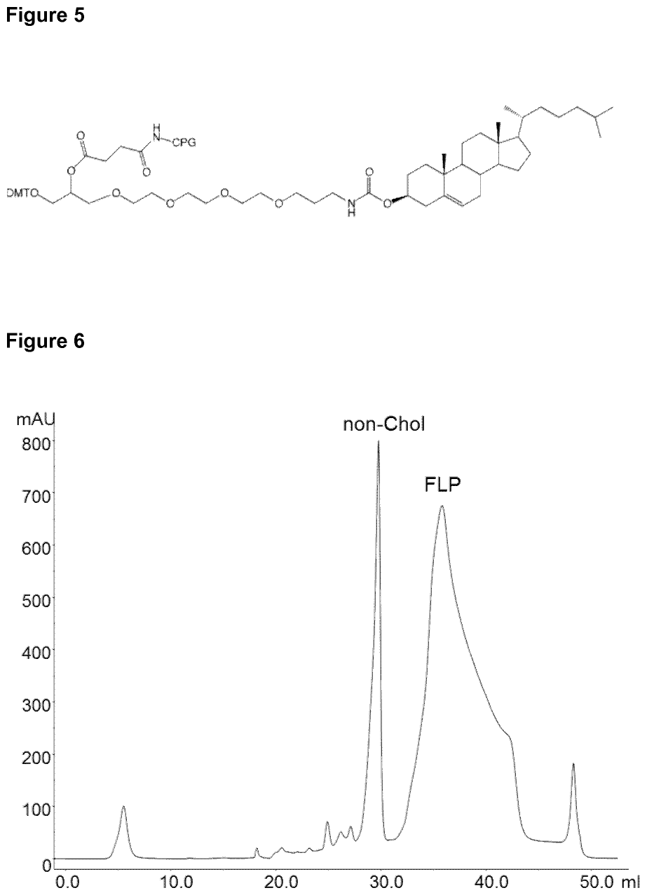

Sequence: A 20mer consisting of alternating RNA nucleotides and 2′-O-Methyl nucleotides was extended by a DNA nucleotide and a cholesterol ligand on the 3′-end. The sequence was assembled on a controlled pore glass (CPG) solid support loaded with cholesterol. The pore size was 500A and the cholesterol loading was 85 μmol/g. The solid support was obtained from Prime Synthesis (Aston, Pa. 19014, USA). The structure of the immobilized cholesterol is shown in FIG. 5.

The oligonucleotide sequence was prepared employing the well established phosphoramidite based oligomerization chemistry. RNA phosphoramidites, 2′-O-Methylphosphoramidites as well as ancillary reagents were purchased from SAFC Proligo (Hamburg, Germany). Specifically, the following amidites were used: (5′-O-dimethoxytrityl-N⁶-(benzoyl)-2′-O-t-butyldimethylsilyl-adenosine-3′-O-(2-cyanoethyl-N,N-diisopropylamino) phosphoramidite, 5′-O-d imethoxytrityl-N⁴-(acetyl)-2′-O-t-butyldimethylsilyl-cytidine-3′-O-(2-cyanoethyl-N,N-diisopropylamino) phosphoramidite, (5′-O-d imethoxytrityl-N²-(isobutyryl)-2′-O-t-butyldimethylsilyl-guanosine-3′-O-(2-cyanoethyl-N,N-diisopropylamino) phosphoramidite, and 5′-O-dimethoxytrityl-2′-O-t-butyldimethylsilyl-uridine-3′-O-(2-cyanoethyl-N,N-diisopropylamino) phosphoramidite. 2′-O-Methylphosphoramidites carried the same protecting groups as the regular RNA amidites. All amidites were dissolved in anhydrous acetonitrile (100 mM) and molecular sieves (3 Å) were added. 5-Ethyl thiotetrazole (ETT, 500 mM in acetonitrile) was used as activator solution. Coupling times were 8 minutes for RNA residues and 6 minutes for 2′-O-methyl residues.

The support bound cholesterol conjugated oligonucleotide was cleaved from the solid phase and deprotected according to published procedures (Wincott, F. et al. Synthesis, deprotection, analysis and purification of RNA and ribozymes (Nucleic Acids Res. 23, 2677-2684, 1995). Typical crude materials contained the desired full length product (FLP) in a range of 70-80%.

To investigate different conditions for HPLC purification of the crude cholesterol conjugated oligonucleotide small scale columns with 5 mm diameter and 50 mm bed height were used. These 1 mL columns were packed with anion exchange resins typically used to purify oligonucleotides. Specifically, two different AEX beads were tested. Source 15Q (15 μm beads) available from GE Healthcare and TSKgel SuperQ-5PW (20 μm beads) available from Tosoh were selected. Purifications were carried out on an AKTA Purifier 100 (GE Healthcare).

For elution, the following buffers were used: Buffer A was made of 20 mM Tris, pH 8. Buffer B had the same composition as buffer A, but contained additionally 500 mM sodium perchlorate (NaClO₄) or 1.4 M Sodium bromide (NaBr). Moreover, because of the hydrophobic nature of the cholesterol ligand (each failure sequence is composed of a 3′-cholesterol due to the chemical synthesis starting from the 3′-end) buffers contained 20-30% acetonitrile (ACN) as well.

For purifications at elevated temperatures a column oven (CO30 from Torrey Pines Scientific, Carlsbad, Calif., USA) and a mobile phase pre-heater (TL-600 available from Timberlein instruments, Boulder, Colo., USA) was used. Both devices were set to the same temperature (e.g. 60° C.).

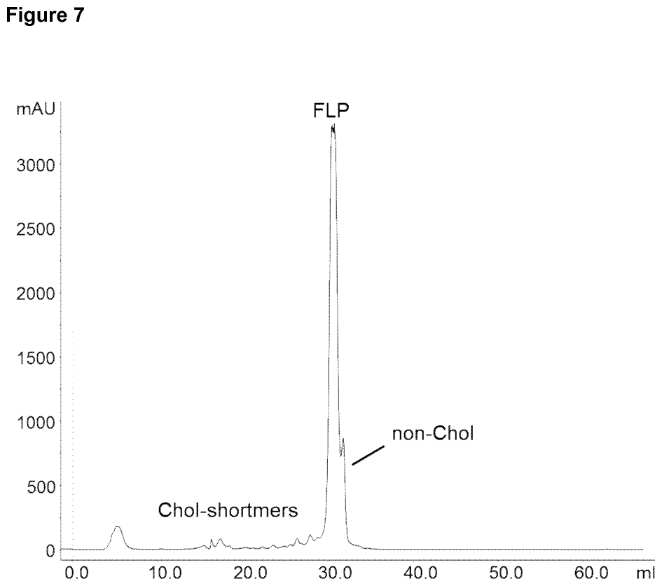

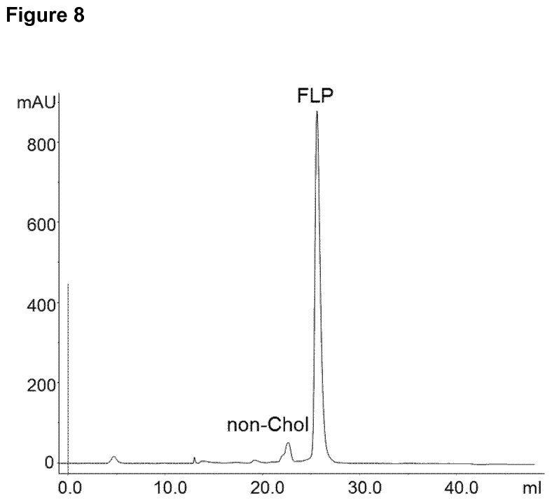

The addition of MbCD to the elution buffers has been demonstrated to alter the elution profile in a predictable manner and enables purifications at ambient temperature as (truncated) cholesterol conjugated oligonucleotides elute in distinct peaks (see FIGS. 6 and 7). When no MbCD was added, a temperature of 60° C. is needed to obtain distinct peaks for cholesterol conjugated oligonucleotides (see FIG. 8).

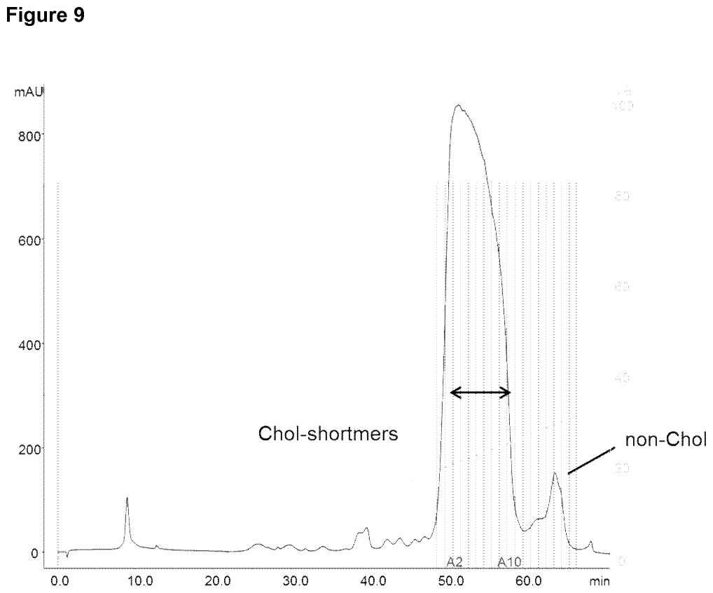

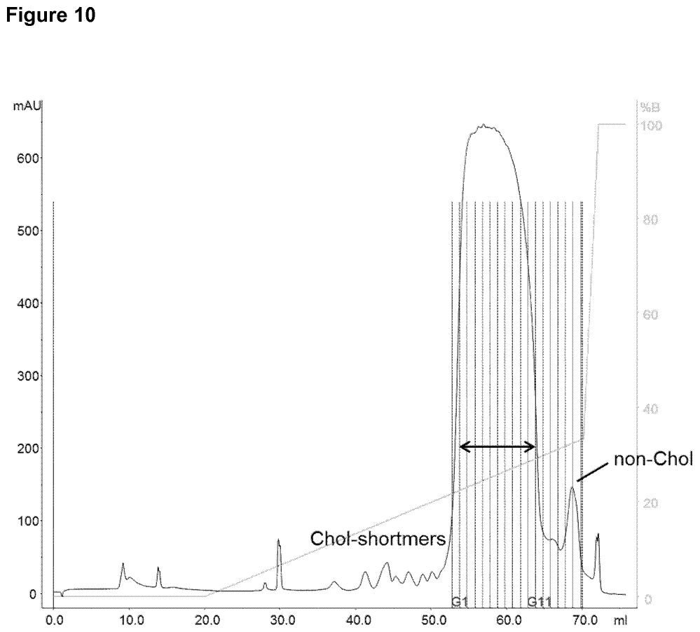

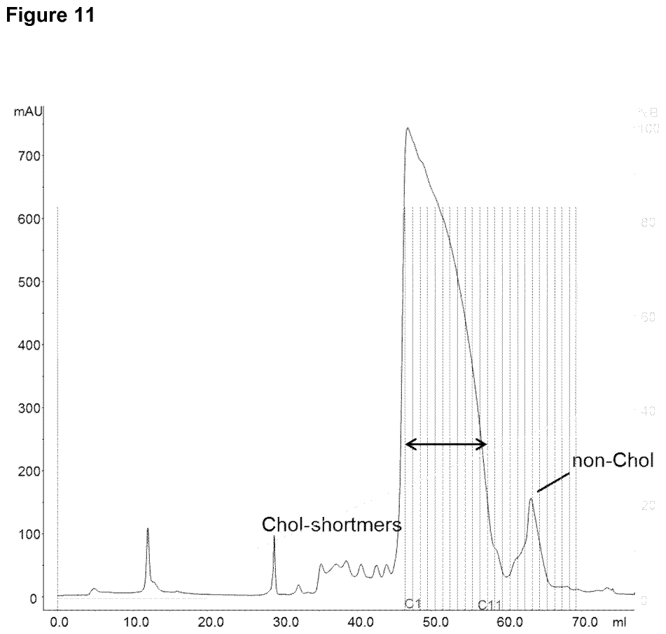

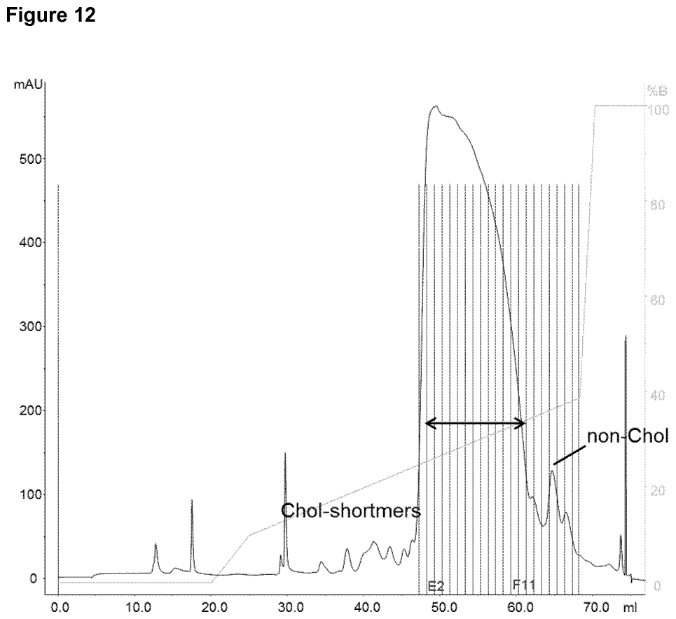

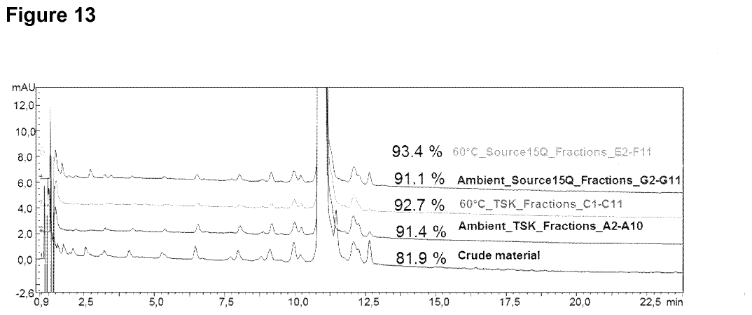

Taken together, the addition of MbCD to the elution buffers allows for IEX HPLC purification of cholesterol conjugated oligonucleotides at ambient temperature (see FIGS. 9 to 12). In addition, the amount of ACN modifier in the mobile phase can be reduced significantly.

These features render capital investments into mobile phase pre-heaters and column ovens or jacketed columns unnecessary. In addition, organic solvents/waste can be cut at least in half.

1. A method for detecting at least one oligonucleotide conjugate of interest in solution, wherein the oligonucleotide conjugate of interest is composed of a nucleic acid entity and of a nonpolar entity, wherein the nucleic acid entity is chemically linked to the nonpolar entity, and wherein the method comprises the steps of:
a) providing a liquid sample comprising the oligonucleotide conjugate of interest;
b) separating the oligonucleotide conjugate of interest from the liquid sample by analytical means under conditions including the presence of at least one cyclodextrin in solution;
c) detecting the oligonucleotide conjugate of interest by means of qualitative or quantitative analysis.

2. The method of claim 1, wherein the analytical means of step b) is selected from the group consisting of anion exchange high performance liquid chromatography (AEX-HPLC), size exclusion liquid chromatography (SEC-LC), reverse phase high performance liquid chromatography (RP-HPLC), ion pairing reversed phase high performance liquid chromatography (IP-RP-HPLC) and capillary gel electrophoresis (CGE).

3. The method of claim 1, wherein the nucleic acid entity of the oligonucleotide conjugate is composed of DNA or RNA nucleotides or any combination thereof, preferably wherein the nucleic acid entity is a chemically synthesized oligonucleotide, more preferably a chemically synthesized oligonucleotide comprising or consisting of modified DNA nucleotides and/or modified RNA nucleotides.

4. The method of claim 1, wherein the nucleic acid entity has a length of from 6 to 150 nucleotides, preferably of from 10 to 80 nucleotides, more preferably of from 12 to 50 nucleotides.

5. The method of claim 1, wherein step c) further includes the detecting of impurities of the oligonucleotide conjugate of interest, preferably wherein the impurities are composed of or consist of at least one non-full length nucleic acid entity, more preferably in the form of one or more non-full length synthesis product(s), even more preferably with a length or structure different to the full-length synthesis product, or any combination thereof.

6. The method of claim 1, wherein the nonpolar entity is a lipophilic or a hydrophobic entity, preferably wherein the nonpolar entity is selected from the group consisting of cholesterol, tocopherol and fluoroquinolone, more preferably wherein the nonpolar entity is cholesterol.

7. The method of claim 1, wherein
i) the anion exchange high performance liquid chromatography (AEX-HPLC) is performed at a temperature of from 10° C. to 90° C., preferably at a temperature of from 30° C. to 75° C., preferably at ambient temperature;
ii) the size exclusion high performance liquid chromatography (SEC-HPLC) is performed at a temperature of from 10° C. to 50° C., preferably at a temperature of from 20° C. to 40° C.;
iii) the reverse phase high performance liquid chromatography (RP-HPLC) is performed at a temperature of from 10° C. to 100° C., preferably at a temperature of from 40° C. to 70° C.;
iv) the ion pairing reverse phase high performance liquid chromatography (IP-RP-HPLC) is performed at a temperature of from 10° C. to 100° C., preferably at a temperature of from 30° C. to 85° C.;
v) the capillary gel electrophoresis (CGE) is performed at a temperature of from 10° C. to 60° C., preferably at a temperature of from 30° C. to 50° C.

8. The method of claim 1, wherein the at least one cyclodextrin is selected from the group consisting of alpha, beta, gamma or delta variants of cyclodextrin, preferably wherein the at least one cyclodextrin is in the form of methyl-beta cyclodextrin.

9. The method of claim 1, wherein the at least one cyclodextrin in solution is present at a final concentration of from 0.01 mM to 50 mM, preferably at a final concentration of from 0.5 mM to 25 mM, more preferably at a final concentration of from 10 mM to 25 mM, most preferred at a final concentration of 20 mM.

10. The method of claim 1, wherein the at least one cyclodextrin is added to the liquid sample before carrying out step b).

11. The method of claim 1, wherein the detecting in step c) is carried out by means of UV readout, by means of fluorescence readout or by means of mass spectrometry (MS), or any method alike.

12. The method of claim 1, wherein the method is used for analytical or preparative purposes, preferably
i) wherein, if the method is used for analytical purposes, the quality of the synthesis product is determined in step c), preferably by determining the degree of impurities; or
ii) wherein, if the method is used for preparative purposes, the yield of the full-length synthesis product is optimized in step c) in that liquid fractions containing the oligonucleotide conjugate of interest are collected.

13. The method of claim 12, wherein, if the method is used for analytical purposes, the quality of the synthesis product is defined by the amount and/or by the ratio of the full-length synthesis product versus the amount and/or the ratio of the non full-length synthesis products, preferably wherein the non full-length synthesis products are intermediate and/or irregular synthesis products or any combination thereof, more preferably wherein the intermediate synthesis products lack one or more nucleotides at either ends or at both ends, most preferably wherein the intermediate synthesis products have the form of n-1, n-2, n-3, n-4, n-5, n-6, n-7, n-8, n-9, n-10, or alike.

14. A method for evaluating the quality of chemically synthesized oligonucleotides, wherein the method comprises the steps of:
a) providing a liquid sample containing or suspected of containing at least one oligonucleotide conjugate of interest, wherein the at least one oligonucleotide conjugate of interest is composed of a nucleic acid entity and of a nonpolar entity, wherein the nucleic acid entity is chemically linked to the nonpolar entity, and wherein the nucleic acid entity is a chemical oligonucleotide synthesis product;
b) separating the at least one oligonucleotide conjugate of interest from the liquid sample by analytical means under conditions including the presence of at least one cyclodextrine in solution;
c) detecting the at least one oligonucleotide conjugate of interest by means of qualitative or quantitative analysis;
d) collecting liquid fractions;
e) analysing the collected fractions containing or suspected of containing the oligonucleotide conjugate of interest, characterized in that the nucleic acid entity of the oligonucleotide conjugate of interest is composed or consists of the at least one full-length synthesis product.

15. (canceled)


SEQUENCE LISTING (2 sequences)

SEQ ID NO: 1 - DNA, 21 bp, Artificial Sequence
  FLPs
  misc_feature (1)..(1)
ggaugaaguggagauuagutt

SEQ ID NO: 2 - DNA, 21 bp, Artificial Sequence
  FLPas
acuaaucuccacuucaucctt